(19) United States
(12) Patent Application Publication
(10) Pub. No.: US 2010/0204244 A1
(43) Pub. Date: Aug. 12, 2010

(54) AGENTS FOR TREATMENT OF DIABETIC RETINOPATHY AND DRUSEN FORMATION IN MACULAR DEGENERATION

(75) Inventors: Robert A. Landers, Arlington, TX (US); David P. Bingaman, Weatherford, TX (US)

Correspondence Address: ALCON, IP LEGAL, TB4-8, 6201 SOUTH FREEWAY, FORT WORTH, TX, 76134, US

(73) Assignee: ALCON INC., Hunenberg (CH)

(21) Appl. No.: 12/715,853

(22) Filed: Mar. 2, 2010

Related U.S. Application Data
(63) Continuation of application No. 11/016,116, filed on Dec. 17, 2004.
(60) Provisional application No. 60/531,811, filed on Dec. 22, 2003.

(51) Int. Cl. A61K 31/497; A61K 31/05; A61K 31/21; A61K 31/122; A61K 31/095; A61K 31/085; A61K 31/352; A61K 31/145; A61K 31/47; A61P 27/02; A61P 25/00

(52) U.S. Cl. 514/255.05; 514/731; 514/506; 514/682; 514/706; 514/718; 514/456; 514/638; 514/311; 514/457

(57) ABSTRACT

Agents that stimulate nuclear translocation of Nrf2 protein and the subsequent increases in gene products that detoxify and eliminate cytotoxic metabolites are provided in a method for treating diabetic retinopathy or drusen formation in age-related macular degeneration. The structurally diverse agents that act on the Nrf2/ARE pathway induce the expression of enzymes and proteins that possess chemically versatile cytoprotective properties and are a defense against toxic metabolites and xenobiotics. Agents include certain electrophiles and oxidants such as a Michael Addition acceptor, diphenol, thiocarbamate, quinone, 1,2-dithiole-3-thione, butylated hydroxyanisole, flavonoid other than genistein, an isothiocyanate, 3,5-di-tert-butyl-4-hydroxytoluene, ethoxyquin, a coumarin, combinations thereof, or a pharmacologically active derivative or analog thereof.

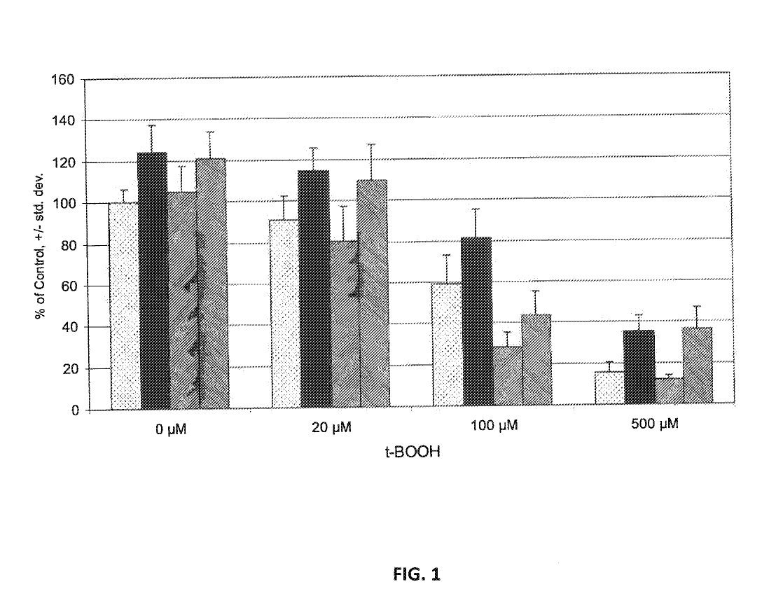

The present application is a continuation of U.S. patent application Ser. No. 11/016,116 filed Dec. 17, 2004, which claims benefit to Provisional Application Ser. No. 60/531,811 filed Dec. 22, 2003.

### FIELD OF THE INVENTION

The present invention relates to the field of prophylactic agents and therapeutics for diabetic retinopathy and drusen formation in age-related macular degeneration.

### BACKGROUND OF THE INVENTION

Diabetic retinopathy is an eye disease that develops in diabetes due to changes in the cells that line blood vessels. When glucose levels are high, as in diabetes, glucose can cause damage in a number of ways. For example, glucose, or a metabolite of glucose, binds to the amino groups of proteins, leading to tissue damage. In addition, excess glucose enters the polyol pathway resulting in accumulations of sorbitol. Sorbitol cannot be metabolized by the cells of the retina and can contribute to high intracellular osmotic pressure, intracellular edema, impaired diffusion, tissue hypoxia, capillary cell damage, and capillary weakening. Diabetic retinopathy involves thickening of capillary basement membranes and prevents pericytes from contacting endothelial cells of the capillaries. Loss of pericytes increases leakage of the capillaries and leads to breakdown of the blood-retina barrier. Weakened capillaries lead to aneurysm formation and further leakage. These effects of hyperglycemia can also impair neuronal functions in the retina. This is an early stage of diabetic retinopathy termed nonproliferative diabetic retinopathy.

Retinal capillaries can become occluded in diabetes causing areas of ischemia in the retina. The non-perfused tissue responds by eliciting new vessel growth from existing vessels (angiogenesis). These new blood vessels can also cause loss of sight, a condition called proliferative diabetic retinopathy, since the new blood vessels are fragile and tend to leak blood into the eye.

Oral administration of genistein, an isoflavonoid found in soybeans, reportedly reduces retinal vascular leakage in experimentally induced diabetic rats (Invest Ophthalmol Vis Sci, 2001, 42, 2110-2114). PCT patent application no. PCT/US02/40457 to Gao, X., et al., published as WO 03/051313, reportedly provides an induction of a phase II detoxification enzyme by sulforaphane in human retinal pigment epithelial cells. However, epithelial cells differ from vascular endothelial cells and biological responses from endothelial tissues to particular therapeutic agents cannot be predicted from the biological responses of epithelial cells.

Given the difficulty in maintaining good glycemic control in human diabetics, development of drugs that inhibit or slow retinal capillary cell and retinal neuron damage would provide a means of reducing the early cellular damage that occurs in diabetic retinopathy.

Macular degeneration is the loss of photoreceptors in the portion of the central retina, termed the macula, responsible for high-acuity vision. Age-related macular degeneration (AMD) is described as either “dry” or “wet.” The wet, exudative, neovascular form of AMD affects about 10% of those with AMD and is characterized by abnormal blood vessels growing through the retinal pigment epithelium (RPE), resulting in hemorrhage, exudation, scarring, or serous retinal detachment. Ninety percent of AMD patients have the dry form characterized by atrophy of the retinal pigment epithelium and loss of macular photoreceptors. At present there is no cure for any form of AMD, although some success in attenuation has been obtained with photodynamic therapy.

Drusen is debris-like material that accumulates with age below the RPE. Drusen is observed using a funduscopic eye examination. Normal eyes may have maculas free of drusen, yet drusen may be abundant in the retinal periphery. The presence of soft drusen in the macula, in the absence of any loss of macular vision, is considered an early stage of AMD. Drusen contains a variety of lipids, polysaccharides, and glycosaminoglycans along with several proteins, modified proteins or protein adducts.

Crabb, J. W., et al. (Proc Natl Acad Sci 99:23, 14682-14687) reportedly provides proteomic analysis of drusen isolated from normal and AMD donor eyes. Protection of cultured human RPE cells from chemical oxidants is reportedly provided by oltipraz, a dithiolethione (Invest Ophthalmol Vis Sci, 2002, 43, 3550-3554), sulforaphane, an isothiocyanate (Proc Natl Acad Sci, 2001, 98, 15221-15226), and dimethylfumarate (Prog Ret Eye Res, 2000, 19, 205-221). However, no suggestion is provided by these references that drusen formation is affected by such treatment.

There is no generally accepted therapeutic method that addresses drusen formation and thereby manages the progressive nature of AMD. In view of the impact of AMD on health and well-being, and the inadequacies of prior methods of treatment, it would be desirable to have an improved method of treatment that addresses early stage AMD, in particular, formation of drusen deposits.

### BRIEF DESCRIPTION OF THE DRAWING

The FIGURE demonstrates Cytoprotective effects of quercetin in retinal endothelial cells exposed to an oxidant stress, t-butyl hydroperoxide. Symbols are as follows: [symbol] control; [symbol] quercetin; ///+buthionine-(S,R)-sulfoximine; \\\+quercetin and buthionine-(S,R)-sulfoximine; *, greater than respective t-BOOH control P<0.001; #, less than respective t-BOOH control P<0.004; ‡, less than zero t-BOOH control P<0.04.

### SUMMARY OF THE INVENTION

According to the present invention, an agent having stimulatory activity for Nrf2 protein nuclear translocation and the subsequent increases in gene products that detoxify and eliminate cytotoxic metabolites provides a protective or therapeutic effect in delaying or preventing retinal vascular and neuronal damage due to diabetic retinopathy. Such agents also provide an inhibitory effect on formation of drusen deposits that accompany macular degeneration. As used herein “stimulatory activity for Nrf2 protein nuclear translocation” means an agent that enhances the availability or the transport of Nrf2 to the nucleus. Translocation of Nrf2 protein to the nucleus allows a subsequent increase in expression of gene products that detoxify and eliminate cytotoxic metabolites.

The methods of the present invention provide a method of treatment for diabetic retinopathy in a subject, the method comprising administering to the subject an effective amount of a composition comprising an agent having stimulatory activity for nuclear translocation of Nrf2 protein, and an acceptable carrier. The subject may be at risk for developing diabetic retinopathy or drusen formation or may have symptoms of diabetic retinopathy or drusen formation. The agent that stimulates nuclear translocation of Nrf2 protein and the subsequent increases in gene products that detoxify and eliminate cytotoxic metabolites of the present invention comprises a Michael Addition acceptor, diphenol, thiocarbamate, quinone, 1,2-dithiole-3-thione, butylated hydroxyanisole, flavonoid other than genistein, an isothiocyanate, 3,5-di-tert-butyl-4-hydroxytoluene, ethoxyquin, a coumarin, combinations thereof, or a pharmacologically active derivative or analog thereof. In one embodiment, the agent comprises an isothiocyanate such as sulforaphane, or a pharmacologically active derivative or analog thereof. In another embodiment, the agent comprises a 1,2-dithiole-3-thione such as oltipraz, or a pharmacologically active derivative or analog thereof.

A further embodiment of the present invention is a method of predicting a therapeutic response of a test agent against diabetic retinopathy in a subject wherein the test agent has stimulatory activity for nuclear translocation of Nrf2 protein. The method comprises exposing a first sample of retinal cells to an oxidative stress, exposing a second sample of retinal cells to the oxidative stress in combination with the test agent; and comparing viable cell number from the exposed first sample to viable cell number from the exposed second sample. When viable cell number from the second sample is greater than the viable cell number from the first sample, the test agent is predicted to provide a therapeutic response to diabetic retinopathy in the subject.

The methods of the present invention further provide a method of inhibiting subretinal drusen formation of a subject, the method comprising administering to the subject an effective amount of a composition comprising an agent having stimulatory activity for Nrf2 protein nuclear translocation, and an acceptable carrier. The agent comprises a Michael Addition acceptor, diphenol, thiocarbamate, quinone, 1,2-dithiole-3-thione, butylated hydroxyanisole, flavonoid, an isothiocyanate, 3,5-di-tert-butyl-4-hydroxytoluene, ethoxyquin, a coumarin, combinations thereof, or a pharmacologically active derivative or analog thereof.

Administration of the agent that stimulates nuclear translocation of Nrf2 protein and the subsequent increases in gene products that detoxify and eliminate cytotoxic metabolites may be by intraocular injection, implantation of a slow release delivery device, or topical, oral, intranasal administration, systemic injection, or other systemic administrations.

In a further embodiment of the present inventive method, the subject is diagnosed with diabetic retinopathy or drusen formation and, in another embodiment of the invention, the subject has symptoms of diabetic retinopathy or drusen formation.

### DETAILED DESCRIPTION OF THE INVENTION

The present invention relates to use of agents that stimulate nuclear translocation of Nrf2 protein and the subsequent increases in gene products that detoxify and eliminate cytotoxic metabolites as a method of treating diabetic retinopathy and drusen formation in age-related macular degeneration.

The term “treating diabetic retinopathy,” as used herein, means delaying or preventing the development of, inhibiting the progression of, or alleviating effects of diabetic retinopathy, or symptoms thereof. Stimulating nuclear translocation of Nrf2 protein and the subsequent increases in gene products that detoxify and eliminate cytotoxic metabolites is provided for protection of retinal vascular capillaries and retinal neurons in a diabetic condition.

The term “treating drusen formation,” as used herein, means delaying or preventing the development of, inhibiting the progression of, or alleviating effects of drusen presence in the subretinal area. Stimulating nuclear translocation of Nrf2 protein and the subsequent increases in gene products that detoxify and eliminate cytotoxic metabolites is provided for protection of the macula by treating drusen formation.

The nuclear translocation of Nrf2 is induced in cells exposed to certain electrophiles and oxidants. Genes induced due to nuclear translocation of Nrf2 yield detoxification enzymes that enhance protection against electrophiles and promote the repair or degradation of damaged proteins. Induction of these enzymes is regulated at the transcriptional level and is mediated by a specific enhancer, the antioxidant response element or ARE, found in the promoter of the gene encoding the enzyme. The sequence context of the ARE, the nature of the chemical inducers, and the cell type affect the activity of the enhancer in a particular gene.

The transcription factor Nrf2 is a member of the NF-E2 transcription factor family and is responsible for upregulating the antioxidant response element (ARE)-mediated gene expression. Nrf2 induces gene expression by binding to the ARE (antioxidant response element) region of the promoter to activate gene transcription constitutively or in response to an oxidative stress signal. Under normal conditions, Nrf2 is thought to be present in the cytoplasm bound by a repressor protein Keap 1, a cytoplasmic protein anchored to the actin cytoskeleton. Not wanting to be bound by theory, it is believed that agents having stimulatory activity for Nrf2 protein nuclear translocation may compete with the cysteine-rich intervening region of a cytosolic factor Keapl for interaction with Nrf2 (Dinkova-Kostova, A. T., et al., Proc Natl Acad Sci, USA, 99:11908-11913 (2002)). Disruption of the

Nrf2-Keapl complex by certain compounds such as sulforaphane may free Nrf2 to translocate into the nucleus where it can heterodimerize with other transcription factors (i.e. Maf, c-Jun, etc.) on ARE regions of genes leading to induction of ARE-regulated gene expression.

Enzymes and proteins expressed by this Nrf2/ARE pathway possess chemically versatile cytoprotective properties and are a defense against toxic metabolites and xenobiotics. Enzymes and proteins known to be expressed through the Nrf2/ARE pathway include glutathione-S-transferases, UDP-glucuronosyltransferases, NADP(H) quinone oxidoreductase, γ-glutamylcysteine synthetase, chaperone/stress response proteins, and ubiquitin/proteasome proteins.

Agents having stimulatory activity for Nrf2 protein nuclear translocation include, for example:
Michael addition acceptors (e.g., α,β-unsaturated carbonyl compounds), such as diethyl maleate or dimethylfumarate;
diphenols such as resveratrol,
butylated hydroxyanisoles such as 2(3)-tert-butyl-4-hydroxyanisole,
thiocarbamates such as pyrrolidinedithiocarbamate,
quinones such as tert-butyl-hydroquinone,
isothiocyanates such as sulforaphane, its precursor glucosinolate, glucoraphanin, or phenethyl isothiocyanate (PEITC),
1,2-dithiole-3-thiones such as oltipraz,
3,5-di-tert-butyl-4-hydroxytoluene,
ethoxyquin,
coumarins such as 3-hydroxycoumarin,
flavonoids such as quercetin or curcumin for treatment of drusen formation, a flavonoid other than genistein such as quercetin or curcumin for treatment of diabetic retinopathy,
diallyl sulfide,
indole-3-carbinol,
epigallo-3-catechin gallate,
ellagic acid,
combinations thereof, or a pharmacologically active derivative or analog thereof.

A Michael acceptor is a molecule that has an alkene adjacent to an electron withdrawing group. The electron withdrawing group is usually a carbonyl, but can also be a nitrile or nitro group. Though chemically diverse, these compounds are electrophiles and have the ability to react with nucleophilic sulfhydryl groups. A “pharmacologically active derivative thereof,” is an agent structurally related to any of the above compounds having stimulatory activity for Nrf2 protein nuclear translocation and derivable from it and may be an ester, an amide, or a salt thereof, for example. A “pharmacologically active analog thereof,” is an agent that is structurally similar to any of the above compounds having stimulatory activity for Nrf2 protein nuclear translocation but differs slightly in composition such as in the replacement of one atom by an atom of a different element or in the presence of a particular functional group, for example. In one embodiment, the present invention provides sulforaphane, oltipraz, a pharmacologically active analog thereof, or a pharmaceutically acceptable salt thereof in a method of treatment for diabetic retinopathy or drusen formation related to age-related macular degeneration.

Sulforaphane (Product no. S6317, Sigma-Aldrich) is known to induce quinone reductase, glutathione-S-transferase, and glutathione reductase, for example. Enzyme induction has been observed in various cell lines including human adult retinal pigment epithelial cells (Zhang, Y. et al., Proc Natl Acad Sci, USA, 89:2399-2403 (1992)). Sulforaphane analogs include, for example, 6-(isothiocyanato-2-hexanone), exo-2-acetyl-6-isothiocyanatonorbornane, exo-2-(isothiocyanato-6-methylsulfonylnorbornane), 6-isothiocyanato-2-hexanol, 1-(isothiocyanato-4-dimethylphosphonylbutane, exo-2-(1-hydroxyethyl)-5-) isothiocyanatonorbornane, exo-2-acetyl-5-isothiocyanatonorbornane, 1-(isothiocyanato-5-methylsulfonylpentane), cis-3-(methylsulfonyl)(cyclohexylmethylisothiocyanate) and trans-3-(methylsulfonyl)(cyclohexylmethylisothiocyanate).

The term “oxidative stress,” as used herein, means exposure to an agent that effects elevated levels of reactive oxygen species (ROS) such as superoxide radicals, hydroxyl ion radicals, hydrogen peroxide, singlet oxygen, or lipid peroxides, for example. Oxidative stress is achieved by inducing physiological conditions that promote the generation of ROS and by the impairment of cellular antioxidant systems, which has been shown in experimental diabetic rats, experimental galactosemic rats, Nrf2 deficient mice, and as a consequence of the aging process.

In cell culture systems, oxidative stress is also induced by the generation or addition of ROS or by the inhibition of antioxidant systems. For example, hydrogen peroxide and t-butyl hydroperoxide can be added to culture media. Menadione can be added to provide a source of superoxide. 4-Hyroxynonenal is an end product of lipid peroxidation that can be included in media, and peroxynitrite can be generated from nitric oxide donors in the presence of superoxide. Buthionine-(S,R)-sulfoximine inhibits the synthesis of glutathione, an important cellular antioxidant. In addition, cells maintained under high glucose or in the presence of advanced glycation end products will increase production of endogenous ROS.

Further, ischemic hypoxia and reperfusion can be employed in both animal models and cell and organ culture systems to impose oxidative stress on biological systems, for example.

The term “retinal cells,” as used herein, includes endothelial cells, neurons, glia, or pericytes, for example.

Mode of administration: The agents of the present invention may be delivered directly to the eye (for example: topical ocular drops or ointments; slow release devices in the cul-de-sac or implanted adjacent to the sclera or within the eye; periocular, conjunctival, sub-tenons, intracameral, intravitreal, or intracanalicular injections) or systemically (for example: orally, intravenous, subcutaneous or intramuscular injections; parenterally, dermal or nasal delivery) using techniques well known by those skilled in the art. It is further contemplated that the agents of the invention may be formulated in intraocular insert or implant devices.

Subject: A subject treated for diabetic retinopathy or drusen formation as described herein may be a human or another animal at risk of developing diabetic retinopathy or drusen formation leading to age-related macular degeneration or having symptoms of diabetic retinopathy or drusen formation related to age-related macular degeneration.

Formulations and Dosage: The agents of the present invention can be administered as solutions, suspensions, or emulsions (dispersions) in a suitable ophthalmic carrier. The following are examples of possible formulations embodied by this invention.

| | Amount in weight % |
|---|---|
| Agent stimulating Nrf2 protein | 0.01-5; 0.01-2.0; 0.5-2.0 |
| nuclear translocation | |
| Hydroxypropylmethylcellulose | 0.5 |
| Sodium chloride | .8 |
| Benzalkonium Chloride | 0.01 |
| EDTA | 0.01 |
| NaOH/HCl | qs pH 7.4 |
| Purified water | qs 100% |

| | Amount in weight % |
|---|---|
| Agent stimulating Nrf2 protein | 0.00005-0.5; 0.0003-0.3; |
| nuclear translocation | 0.0005-0.03; 0.001 |
| Phosphate Buffered Saline | 1.0 |
| Benzalkonium Chloride | 0.01 |
| Polysorbate 80 | 0.5 |
| Purified water | q.s. to 100% |

| | Amount in weight % |
|---|---|
| Agent stimulating Nrf2 protein | 0.001 |
| nuclear translocation | |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water | q.s. to 100% |

| | Amount in weight % |
|---|---|
| Agent stimulating Nrf2 protein | 0.0005 |
| nuclear translocation | |
| Phosphate Buffered Saline | 1.0 |
| Hydroxypropyl-β-cyclodextrin | 4.0 |
| Purified water | q.s. to 100% |

In a further embodiment, the ophthalmic compositions are formulated to provide for an intraocular concentration of about 0.1-100 nanomolar (nM) or, in a further embodiment, 1-10 nM. Peak plasma concentrations of up to 20 micromolar may be achieved for systemic administration. Topical compositions are delivered to the surface of the eye one to four times per day according to the routine discretion of a skilled clinician. The pH of the formulation should be 4-9, or 4.5 to 7.4. Systemic formulations may contain about 10 mg to 1000 mg, about 10 mg to 500 mg, about 10 mg to 100 mg or to 125 mg, for example, of the agent that stimulates nuclear translocation of Nrf2 protein and the subsequent increases in gene products that detoxify and eliminate cytotoxic metabolites

An “effective amount” refers to that amount of agent that is able to stimulate nuclear translocation of Nrf2 protein and the subsequent increases in gene products that detoxify and eliminate cytotoxic metabolites. Such induction of gene expression provides a defense against the toxicity of reactive electrophiles as well as other toxic metabolites. Therefore, an agent that stimulates nuclear translocation of Nrf2 protein and the subsequent increases in gene products that detoxify and eliminate cytotoxic metabolites is provided for protection against cytotoxicity. Such protection delays or prevents onset of symptoms in a subject at risk for developing diabetic retinopathy or drusen formation in age-related macular degeneration. The effective amount of a formulation may depend on factors such as the age, race, and sex of the subject, or the severity of the retinopathy or degree of drusen formation, for example. In one embodiment, the agent is delivered topically to the eye and reaches the retina or drusen at a therapeutic dose thereby ameliorating the diabetic retinopathy or drusen formation process.

While the precise regimen is left to the discretion of the clinician, the resulting solution or solutions are preferably administered by placing one drop of each solution(s) in each eye one to four times a day, or as directed by the clinician.

Acceptable carriers: An ophthalmically acceptable carrier refers to those carriers that cause at most, little to no ocular irritation, provide suitable preservation if needed, and deliver one or more agents that stimulate nuclear translocation of Nrf2 protein and the subsequent increases in gene products that detoxify and eliminate cytotoxic metabolites of the present invention in a homogenous dosage. For ophthalmic delivery, an agent that stimulates nuclear translocation of Nrf2 protein and the subsequent increases in gene products that detoxify and eliminate cytotoxic metabolites may be combined with ophthalmologically acceptable preservatives, co-solvents, surfactants, viscosity enhancers, penetration enhancers, buffers, sodium chloride, or water to form an aqueous, sterile ophthalmic suspension, solution, or viscous or semi-viscous gels or other types of solid or semisolid composition such as an ointment. Ophthalmic solution formulations may be prepared by dissolving the agent in a physiologically acceptable isotonic aqueous buffer. Further, the ophthalmic solution may include an ophthalmologically acceptable surfactant to assist in dissolving the agent. Viscosity building compounds, such as hydroxymethyl cellulose, hydroxyethyl cellulose, methylcellulose, polyvinylpyrrolidone, or the like, may be added to the compositions of the present invention to improve the retention of the compound.

In order to prepare a sterile ophthalmic ointment formulation, the agent that stimulates nuclear translocation of Nrf2 protein and the subsequent increases in gene products that detoxify and eliminate cytotoxic metabolites is combined with a preservative in an appropriate vehicle, such as mineral oil, liquid lanolin, or white petrolatum. Sterile ophthalmic gel formulations may be prepared by suspending the agent in a hydrophilic base prepared from the combination of, for example, CARBOPOL®-940 (BF Goodrich, Charlotte, N.C.), or the like, according to methods known in the art for other ophthalmic formulations. VISCOAT® (Alcon Laboratories, Inc., Fort Worth, Tex.) may be used for intraocular injection, for example. Other compositions of the present invention may contain penetration enhancing materials such as CREMOPHOR® (Sigma Aldrich, St. Louis, Mo.) and TWEEN® 80 (polyoxyethylene sorbitan monolaureate, Sigma Aldrich), in the event the agents of the present invention are less penetrating in the eye.

### Example 1

### Agents Having Stimulatory Activity for Nrf2 Protein Nuclear Translocation

Vascular endothelial cells, such as bovine aortic endothelial cells (BAEC, VEC Technologies, Rensselaer, N.Y.), are used to determine those agents having stimulatory activity for Nrf2 protein nuclear translocation. For example, confluent monolayers of bovine aortic endothelial cells are exposed to candidate agents in Dulbecco's modified Eagle's medium with 1% fetal bovine serum for up to 24 hours. Cell lysates, cytosolic extracts, and nuclear extracts are prepared, and immunoblotting performed and quantified as described in Buckley, B. J., et al. (Biochem Biophys Res Commum, 307:973-979 (2003)). Agents that increase the amount of Nrf2 detected in the nuclear fraction as compared to control cells without agent are then tested for activity in endothelial cells mimicking hyperglycemia as set forth in Example 2.

### Example 2

### Protection of Cells that Mimic Hyperglycemia

Bovine retinal endothelial cells (BREC's) cultured under conditions mimicking hyperglycemia are combined with an agent having stimulatory activity for Nrf2 protein nuclear translocation, then the exposed cells are tested for protection of effects of hyperglycemia by measuring extent of formation of lipid peroxides, or by measuring levels of expression of intercellular cell adhesion molecule-1 (ICAM-1), for example, as described below. A lower extent of formation of lipid peroxides, or a lower level of expression of ICAM-1 in test cultures as compared to a control culture without agent indicates that the agent provides protection from the effects of hyperglycemia.

Assay for formation of lipid peroxides: Isolated bovine retinal microvessel endothelial cells (BRMEC's, VEC Technologies, Rensselaer, N.Y.) are treated or pretreated with an agent having stimulatory activity for Nrf2 protein nuclear translocation. The agent is optionally removed. The treated cells are exposed to 25 mM D-glucose in culture media for up to ten days either prior to, during, or after exposure to the agent. The formation of lipid peroxides in the cells is measured with a commercially available kit (Lipid Hydroperoxide Assay Kit #705002, Cayman Chemical Co., Ann Arbor, Mich.), and compared to that observed in cells exposed to normal (5 mM) D-glucose. A lowered extent of formation of lipid peroxides in cells exposed to the agent as compared with cells not exposed to the agent indicates that the agent provides protection from the effects of hyperglycemia and that the agent is useful for treatment of diabetic retinopathy.

Assay for Protection against Oxidants using Lactate Dehydrogenase Activity: Isolated BRECs are treated or pretreated with an agent having stimulatory activity for Nrf2 protein nuclear translocation. The treated cells are exposed to the stress of oxidants such as t-butylhydroperoxide (up to 0.5 mM) or menadione (up to 0.25 mM), for example, for up to 24 hours. Cell survival is determined by measuring lactate dehydrogenase activity (LDH) release into the culture media due to cell lysis and/or LDH activity retained in the viable cells in a culture exposed to the agent as compared to cells not exposed to the agent. A lowered amount of release of LDH into the media as compared to a control culture not exposed to agent indicates cell survival, that the agent provides protection from the effects of the oxidants, and that the agent is useful for treatment of diabetic retinopathy.

Assay for ICAM-1 Levels: Isolated BRECs are treated or pretreated with an agent having stimulatory activity for Nrf2 protein nuclear translocation. The treated cells are exposed to methylglyoxal (MG) and/or MG-modified BSA as an in vitro model of hyperglycemia in which increases in the expression of intercellular cell adhesion molecule-1 (ICAM-1) occurs as a result of the hyperglycemia. Increased ICAM-1 levels promote the adhesion of leukocytes to vascular endothelium (leukostasis) and lead to capillary nonperfusion. Levels of ICAM-1 are measured by an enzyme-linked immunosorbent assay (ELISA) using commercially available anti-ICAM-1 antibodies from BioVendor (Brno, Czech Republic; Heidelberg, Germany, #RE 11228C100, monoclonal antibody to ICAM-1 from clone MEM-111) or from Zymed Laboratories (South San Francisco, Calif.; MY-13 monoclonal anti-ICAM-1, Buras et al., Am J Cell Phys 278:C292-C302, (2000)). A lowered level of ICAM-1 as compared to a control culture not exposed to agent indicates that the agent provides protection from the effects of the hyperglycemia and that the agent is useful for treatment of diabetic retinopathy.

In the above assays, an agent having stimulatory activity for Nrf2 protein nuclear translocation, such as sulforaphane, oltipraz, or other Nrf2 pathway inducer, may be provided as a treatment or as a pretreatment relative to the hyperglycemic condition or oxidative stress.

### Example 3

### In Vivo Protective Effects of Agents Having Stimulatory Activity for Nrf2 Protein Nuclear Translocation

Retinal vascular permeability in a streptozotocin-induced diabetic rat receiving an agent having stimulatory activity for Nrf2 protein nuclear translocation is tested and compared with the retinal vascular permeability in such a rat not receiving the agent. The method is modified from Nakajima, M., et al. (Investigative Ophthalmology & Visual Science 42:9, August, 2001, pg. 2110-2114). Briefly, a nondiabetic control group of rats, a diabetic control group of rats, and a diabetic group of rats receiving an agent having stimulatory activity for Nrf2 protein nuclear translocation are analyzed for retinal vascular permeability by looking at albumin in extracellular space after perfusion. Diabetes is induced by streptozotocin injection. Retinal vascular permeability is measured using a Western blot analysis for extravasated albumin. Retinal phosphotyrosine levels and proliferating cell nuclear antigen (PCNA) may also be evaluated by Western blot analysis. A lowered level of permeability, i.e., less extravasated albumin, in the agent-treated diabetic group of rats as compared to the diabetic control group of rats indicates that the agent provides protection from the effects of the hyperglycemia and that the agent is useful for treatment of diabetic retinopathy.

### Example 4

### Inhibition of Drusen Formation by Agents Having Stimulatory Activity for Nrf2 Protein Nuclear Translocation

Cultured human retinal pigment epithelium (RPE) cells from the ARPE-19 cell line (ATCC CRL-2502) are treated with an agent that stimulates the nuclear translocation of Nrf2. Treatment with the agent may precede or be concomitant with exposure of the RPE cells to conditions of oxidative stress. The oxidative stress is generated by inclusion of t-butylhydroperoxide (ICN Biomedicals, Irvine, Calif.), menadione (Sigma-Aldrich, St. Louis, Mo.), or S-nitroso-N-acetyl-DL-penicillamine (SNAP, Sigma-Aldrich), or a combination of these agents, in the incubation media for up to seven days Inhibition of the rate-limiting enzyme in glutathione synthesis, γ-glutamylcysteine synthetase, by inclusion of buthionine sulfoximine (Sigma-Aldrich)in the culture medium, is employed alone or in combination with the other agents above. During the course of the treatments, quantitative immunoassays are performed to monitor the formation of malondialdehyde (MDA), 4-hydroxynonenal (HNE), nitrotyrosine, and advanced glycation end product (AGE) modifications to RPE cellular proteins. These assays employ anti-MDA and anti-HNE antisera (Alpha Diagnostics, San Antonio, Tex.), anti-AGE antibody (Wako Chemicals, Richmond, Va.) and anti-nitrotyrosine antibody (Upstate Biotechnology, Lake Placid, N.Y.). A reduction in the level of one or more of the protein modifications in cells exposed to the agent compared to cells not exposed to the agent indicates an inhibition of the formation of protein modifications or adducts found in drusen due to increased expression of genes encoding antioxidant enzymes and proteins and phase II detoxification enzymes.

### Example 5

### In Vitro Oxidant Stress Assay; Protective Effects of Quercetin Pretreatment

The present example provides a study wherein cultured retinal endothelial cells were exposed to an oxidant stress to evaluate the protective effects of pretreatment with quercetin. In addition, the present example provides an assay system for screening compounds for therapeutic activity in nonproliferative diabetic retinopathy.

Bovine retinal endothelial cells (BRECs) (VEC Technologies, Rensselaer, N.Y.) were grown on fibronectin (50 mg/ml)-coated plasticware at 37° C. in 5% CO₂ in MCDB-131 Complete media (VEC Technologies, Rensselaer, N.Y.). For serum-free media (SFM) conditions, MCDB-131 was used supplemented at 5 ml/500 ml with 100× antibiotic/antimycotic, 10 mM L-glutamine, and 0.1% BSA (all from Life Technologies Inc., Grand Island, N.Y.).

The BRECs were seeded at 10,000 cells/well and allowed to attach and grow for three days in 0.2 ml/well in complete media (10% fetal bovine serum (FBS)). The complete media was replaced with SFM for the next twenty-four hours at which time 25 μM quercetin, 100 μM DL-buthionine-(S,R)-sulfoximine (BSO, Sigma, St. Louis, Mo.), and the 0.1% DMSO were added. The next day all media was replaced with SFM (0.1 ml/well) containing 0-500 μM t-butyl hydroperoxide (ICN Biomedicals, Irvine, Calif.). After four hours incubation at 37° C. in 5% CO₂ the assay was started by adding 20 μl of a mixture of twenty parts MTS (3-(4,5-dimethythiazol-2-yl)-5-(3-carboxymethoxyphenyl)-2-(4-sulfophenyl)-2H-tetrazolium, inner salt) and one part PMS (phenazine methosulfate) from the Promega ClelTiter 96® AQᵤₑₒᵤₛ Non-Radioactive Cell Proliferation Assay kit (Promega Corporation, Madison, Wis.). The plates were returned to the incubator for three hours and then net absorbances at 490 nm, obtained by subtraction of a blank mean, were recorded and used as a measure of levels of viable cells.

Using six wells per condition, the assay was carried out three times. The absorbance values were normalized such that the control wells without t-butyl hydroperoxide equaled 100%, and data from the three assays were pooled. An overall statistical difference was found among treatment groups (see FIGURE; ANOVA, P<0.05). As shown by the data of the FIGURE, exposure to t-butyl hydroperoxide resulted in significant reductions in cell survival at all concentrations. Further, pretreatment with quercetin alone yielded higher numbers of viable cells than in the control group for each t-butyl hydroperoxide concentration. Pretreatment with BS alone had no effect on cell survival, but significantly enhanced the toxicity of all t-butyl hydroperoxide concentrations. Combined pretreatment with quercetin and BSO resulted in cell survival equal to that seen with quercetin pretreatment alone with the exception of the 100 μM t-butyl hydroxperoxide exposure group. In that group the combined pretreatment only partially restored cell survival.

In this study, BSO pretreatment enhanced the toxicity of subsequent t-butyl hydroperoxide exposures. This result is to be expected since t-butyl hydroxperoxide is largely eliminated by glutathione peroxidase and since BSO inhibits γ-glutamylcysteine synthetase, the rate-limiting enzyme in glutathione synthesis. Quercetin has been reported to increase glutathione levels by transactivation of the promoter of the catalytic subunit of γ-glutamylcysteine synthetase (Myhrstad, et al., 2002, Free Radical Biology and Medicine, 32:386-393). The decrease of toxicity enhancement of BSO by the combined pretreatment with quercetin and BSO is consistent with a mechanism whereby quercetin has antioxidant effects through enzyme expression via the Nrf2/ARE pathway.

The references cited herein, to the extent that they provide exemplary procedural or other details supplementary to those set forth herein, are specifically incorporated by reference.

Those of ordinary skill in the art, in light of the present disclosure, will appreciate that modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

As used herein and unless otherwise indicated, the terms “a” and “an” are taken to mean “one”, “at least one” or “one or more”.

What is claimed is:

1. A method of preventing retinal vascular and neuronal damage due to diabetic retinopathy in a patient, said method comprising administering to said subject an effective amount of a composition comprising an agent capable of translocating Nrf2 protein to a retinal cell's nucleus wherein expression of gene products that detoxify and eliminate cytotoxic metabolites is activated, and an acceptable carrier,
wherein the agent comprises a Michael Addition acceptor, diphenol, thiocarbamate, quinone, 1,2-dithiole-3-thione, butylated hydroxyanisole, flavonoid other than genistein, an isothiocyanate, 3,5-di-tert-butyl-4-hydroxytoluene, ethoxyquin, a coumarin, or combinations thereof.

2. The method of claim 1, wherein the subject has symptoms of diabetic retinopathy.

3. The method of claim 1, wherein the agent comprises an isothiocyanate.

4. The method of claim 3, wherein the isothiocyanate comprises sulforaphane.

5. The method of claim 1, wherein the agent comprises a 1,2-dithiole-3-thione.

6. The method of claim 5, wherein the 1,2-dithiole-3-thione comprises oltipraz.

7. The method of claim 1, wherein the administering is by intraocular injection, implantation of a slow release delivery device, or topical, oral, or intranasal administration.

8. The method of claim 1, wherein the administering is by intraocular administration.

9. A method of preventing retinal vascular and neuronal damage due to diabetic retinopathy in a subject, said method comprising diagnosing a subject with diabetic retinopathy, and
administering to said subject an effective amount of a composition comprising an agent capable of translocating Nrf2 protein to a retinal cell's nucleus wherein expression of gene products that detoxify and eliminate cytotoxic metabolites is activated, and an acceptable carrier,
wherein the agent comprises a Michael Addition acceptor, diphenol, thiocarbamate, quinone, 1,2-dithiole-3-thione, butylated hydroxyanisole, flavonoid other than genistein, an isothiocyanate, 3,5-di-tert-butyl-4-hydroxytoluene, ethoxyquin, a coumarin, or combinations thereof.

10. A method of inhibiting subretinal drusen formation of a subject, the method comprising:
administering to the subject an effective amount of a composition comprising an agent having stimulatory activity for Nrf2 protein nuclear translocation, and an acceptable carrier,
wherein the agent comprises a Michael Addition acceptor, diphenol, thiocarbamate, quinone, 1,2-dithiole-3-thione, butylated hydroxyanisole, flavonoid, an isothiocyanate, 3,5-di-tert-butyl-4-hydroxytoluene, ethoxyquin, a coumarin, combinations thereof, or a pharmacologically active derivative or analog thereof.

11. The method of claim 10 wherein the subject is at risk for developing subretinal drusen formation.

12. The method of claim 10 wherein the subject has symptoms of developing subretinal drusen formation.

13. The method of claim 10 wherein the agent comprises an isothiocyanate, or a pharmacologically active derivative thereof.

14. The method of claim 13 wherein the isothiocyanate comprises sulforaphane, or a pharmacologically active derivative thereof.

15. The method of claim 10 wherein the agent comprises a 1,2-dithiole-3-thione, or a pharmacologically active derivative thereof.

16. The method of claim 15 wherein the 1,2-dithiole-3-thione comprises oltipraz, or a pharmacologically active derivative thereof.

17. The method of claim 10, wherein the administering is by intraocular injection, implantation of a slow release delivery device, or topical, oral, or intranasal administration.

18. The method of claim 10 wherein the administering is by intraocular administration.

19. A method of treatment for subretinal drusen formation of a subject, the method comprising:
diagnosing a subject with subretinal drusen formation, and
administering to the subject an effective amount of a composition comprising an agent having stimulatory activity for Nrf2 protein nuclear translocation, and an acceptable carrier,
wherein the agent comprises a Michael Addition acceptor, diphenol, thiocarbamate, quinone, 1,2-dithiole-3-thione, butylated hydroxyanisole, flavonoid, an isothiocyanate, 3,5-di-tert-butyl-4-hydroxytoluene, ethoxyquin, a coumarin, combinations thereof, or a pharmacologically active derivative or analog thereof.

20. The method of claim 19 wherein the agent comprises a flavonoid other than genistein.

21. The method of claim 20 wherein the agent comprises quercetin.

22. The method of claim 19 wherein the agent comprises a flavonoid other than genistein.

23. The method of claim 22 wherein the agent comprises quercetin.
